SEQUENCE LISTING (7 sequences)

SEQ ID NO: 1 - DNA, 1758 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 2 - protein, 585 aa, Homo sapiens [sequence omitted]

SEQ ID NO: 3 - DNA, 39 bp, Artificial Sequence
  Forward Primer
tctgtcgacaaaagaaggggtgtgtttcgtcgagatgca

SEQ ID NO: 4 - DNA, 39 bp, Artificial Sequence
  Reverse Primer
atggaattcatgttataagcctaaggcagcttgacttgc

SEQ ID NO: 5 - DNA, 1776 bp, Artificial Sequence
  Nucleotide Sequence of Pro-Human Serum Albumin Gene
aggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 6 - protein, 591 aa, Artificial Sequence
  Amino acid Sequence of the Pro-Human Serum Albumin
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 7 - DNA, 6 bp, Artificial Sequence
  Modified XhOI restriction site
ctcgac

(19) United States
(12) Patent Application Publication
(10) Pub. No.: US 2022/0119494 A1
(43) Pub. Date: Apr. 21, 2022

(54) RECOMBINANT VECTOR, HOST CELL AND PROCESS FOR PRODUCTION OF HUMAN SERUM ALBUMIN

(71) Applicant: SUDERSHAN BIOTECH PVT LTD, Telangana (IN)

(72) Inventors: Mythili K PADALA, Telangana (IN); Ramareddy V GUNTAKA, Telangana (IN)

(73) Assignee: SUDERSHAN BIOTECH PVT LTD, Telangana (IN)

(21) Appl. No.: 17/293,794

(22) PCT Filed: Sep. 17, 2019
(86) PCT No.: PCT/IB2019/057803

§ 371 Date: May 13, 2021

(30) Foreign Application Priority Data
Nov. 14, 2018 (IN) 201841042911

(51) Int. Cl. C07K 14/765; C12N 15/86

(52) U.S. Cl. CPC C07K 14/765; A61K 38/00; C12N 15/86

(57) ABSTRACT

A recombinant vector, host cell and process for production of human serum albumin. The disclosure represents an advancement in the field of genetic engineering and discloses a modified pPIC9 vector comprising a nucleic acid (SEQ ID NO:5) encoding human serum albumin for achieving optimum expression of human serum albumin in Pichia pastoris host cell. The disclosure also discloses a modified process for producing recombinant human serum albumin.

### PRIORITY CLAIM

The present application is a National Phase entry of PCT Application No. PCT/IB2019/057803, filed Sep. 17, 2019 which claims priority from Indian Patent Application number 201841042911, filed on Nov. 14, 2018, each of which is hereby incorporated by reference herein in its entirety.

### FIELD OF INVENTION

The present disclosure relates to the field of genetic engineering. More specifically, the disclosure is directed towards obtaining improved production of recombinant human serum albumin as secreted protein.

### BACKGROUND

Human serum albumin is the most abundant human blood plasma protein, making up about 60% of total plasma proteins. The average concentration of albumin in blood is 40 mg/ml. Human serum albumin helps in maintaining osmotic pressure and performs several other functions such as binding and transport of copper, nickel, calcium, bilirubin, protoporphyrin, long-chain fatty acids, prostaglandins, steroid hormones (weak binding with these hormones promotes their transfer across the membranes), thyroxine, tri-iodothyronine, cysteine and glutathione.

Large amounts of human serum albumin (HSA) are used clinically for treatment of burns, shock and blood loss. Human serum albumin is also used in pharmaceutical preparations such as drug formulations and vaccines. Further, human serum albumin is also used in cell culture media.

At present, fractionated human-donated blood remains the major commercial source for human serum albumin. Fractionated blood contains the risk of transmitting blood-borne contaminants and pathogens. To meet the demand of human serum albumin while avoiding the risk of the presence of pathogenic viruses, it is essential to develop alternate methods for commercial production of human serum albumin.

Recombinant DNA technology has provided promising alternatives for production of human serum albumin. Yeast hosts like Saccharomyces cerevisiae, Kluyveromyces lactis and Pichia pastoris have been used for expression of various genes for the purposes of achieving extra-cellular and enhanced expression of soluble proteins.

Genetically engineered Pichia pastoris has been used in art wherein recombinant human serum albumin structural gene has been isolated, manipulated and inserted in a vector. Specifically, prior art discloses the use of a native gene encoding full length pre-pro Human Serum Albumin (HSA) protein of 609 amino acids having an 18 amino acid pre-domain (MKWVTFISLLFLFSSAYS), 6 amino acid pro-domain (RGVFRR) and 585 amino acid human serum albumin protein. The 18 amino acid pre-domain is processed in the endoplasmic reticulum and the 6 amino acid pro-domain is processed in the Golgi apparatus to secrete the 585 amino acid human serum albumin.

However, the teachings in the art are known to have one or more deficiencies such as poor expression of the HSA gene, poor secretion of the mature HSA protein, slow growth of the yeast during fermentation etc. These factors lead to lower yield and are not sustainable for commercial-scale production of recombinant human serum albumin. Hence, it is desirable to maximize HSA production.

The inventors have developed a strategy for enhancing the yield of biologically active human serum albumin (HSA) protein by expressing HSA gene not encoding the 18 amino acid pre-domain. The removal of the 18 amino acid pre-domain in combination of other factors, such as choice of vector, host cell and modification of process parameters has resulted in an unprecedented enhanced technical effect in terms of yield.

Thus, the present disclosure is aimed at obtaining large-scale production of recombinant human serum albumin. The present disclosure also holds economic significance as it would directly affect the cost of production, making human serum albumin more affordable and widely accessible.

### SUMMARY OF THE INVENTION

### Technical Problem

The technical problem to be solved in this disclosure is to improve the yield of recombinant human serum albumin.

### Solution to the Problem

The problem has been solved by developing a pPIC9 expression vector containing a nucleic acid (SEQ ID NO: 5), which does not encode the 18 amino acid pre-domain of human serum albumin gene. The expression vector has been further modified to contain a mutated XhoI restriction site (CTCGAC).

The pPIC9 vector is used for developing recombinant Pichia pastoris host cells expressing recombinant human serum albumin (rHSA) as secreted protein. Additionally, the fermentation strategy has been modified to obtain a high yield of 1.4-1.7 gm/L recombinant human serum albumin with recoveries in the range of 60-70% and purity of about 98-99%.

### Overview of the Invention

The present disclosure relates to a pPIC9 expression vector comprising a nucleic acid (SEQ ID NO:5) encoding human serum albumin. The expression vector has been further modified to contain a mutated XhoI restriction site. The present disclosure invention also relates to recombinant Pichia pastoris host cells comprising the modified expression vector and used for efficient production of recombinant human serum albumin.

The disclosure also relates to a process for expression of recombinant human serum albumin as secreted protein. The human serum albumin concentration is found to be in the range of 1.4-1.7 gm/L, with recoveries in the range of 60-70% and the purity is about 98-99%.

### OBJECT OF THE INVENTION

The object of the disclosure is to provide a modified vector and a recombinant host cell for optimum production of human serum albumin as secreted protein. A further objective of the disclosure is to provide an efficient process for over-expression and commercial scale production of recombinant human serum albumin (rHSA) in soluble form as a secreted protein.

### BRIEF DESCRIPTION OF DRAWINGS

The features of the present disclosure will become fully apparent from the following description taken in conjunction with the accompanying figures. With the understanding that the figures depict only several embodiments in accordance with the disclosure and are not to be considered limiting of its scope, the disclosure will be described further through use of the accompanying figures.

FIG. 1 represents the construction scheme of pPIC9-HSA expression construct.

FIG. 2 depicts photomicrograph of SDS-PAGE: Expression of secreted rHSA at different time points stained with coomassie brilliant blue. Lane 1: Bovine serum Albumin standard (Sigma); Lane 2: Fermentation broth sample—Uninduced; Lane 3: Fermentation broth sample at 24 hrs post induction; Lane 4: Fermentation broth sample at 48 hrs post induction; Lane 5: Fermentation broth sample at 72 hrs post induction; Lane 6: Fermentation broth sample at 96 hrs post induction; Lane 7: Bovine serum Albumin standard (Sigma).

FIG. 3 depicts elution profile of rHSA from DEAE sepharose column: Peak 1—Eluted fraction from linear gradient of 14.4% to 32% of 0.3 M NaCl. Peak 2—Eluted fraction from linear gradient of 33% to 42% of 0.3M NaCl. Peak 3-Eluted fraction from linear gradient of 43.3% to 80% of 0.3 M NaCl. Peak 4—Eluted fraction of 100% of 0.3M NaCl.

FIG. 4 depicts photomicrograph of native SDS-PAGE of rHSA after DEAE Sepharose column purification: Lane 1—DEAE sepharose column before loading; Lane 2—Flow through sample; Lane 3—peak 1 pooled fractions eluted from DEAE sepharose column; Lane 4—peak 2 pooled fractions eluted from DEAE sepharose column; Lane 5—peak 3 pooled fractions eluted from DEAE sepharose column.

BRIEF DESCRIPTION OF SEQUENCES AND SEQUENCE LISTING

SEQ ID NO: 1 - Nucleotide Sequence of the gene encoding secreted Human Serum
Albumin (1758 base pairs)
GAT GCA CAC AAG AGT GAG GTT GCT CAT CGG TTT AAA GAT TTG GGA GAA
GAA AAT TTC AAA GCC TTG GTG TTG ATT GCC TTT GCT CAG TAT CTT CAG
CAG TGT CCA TTT GAA GAT CAT GTA AAA TTA GTG AAT GAA GTA ACT GAA
TTT GCA AAA ACA TGT GTT GCT GAT GAG TCA GCT GAA AAT TGT GAC AAA
TCA CTT CAT ACC CTT TTT GGA GAC AAA TTA TGC ACA GTT GCA ACT CTT
CGT GAA ACC TAT GGT GAA ATG GCT GAC TGC TGT GCA AAA CAA GAA CCT
GAG AGA AAT GAA TGC TTC TTG CAA CAC AAA GAT GAC AAC CCA AAC CTC
CCC CGA TTG GTG AGA CCA GAG GTT GAT GTG ATG TGC ACT GCT TTT CAT
GAC AAT GAA GAG ACA TTT TTG AAA AAA TAC TTA TAT GAA ATT GCC AGA
AGA CAT CCT TAC TTT TAT GCC CCG GAA CTC CTT TTC TTT GCT AAA AGG
TAT AAA GCT GCT TTT ACA GAA TGT TGC CAA GCT GCT GAT AAA GCT GCC
TGC CTG TTG CCA AAG CTC GAT GAA CTT CGG GAT GAA GGG AAG GCT TCG
TCT GCC AAA CAG AGA CTC AAG TGT GCC AGT CTC CAA AAA TTT GGA GAA
AGA GCT TTC AAA GCA TGG GCA GTA GCT CGC CTG AGC CAG AGA TTT CCC
AAA GCT GAG TTT GCA GAA GTT TCC AAG TTA GTG ACA GAT CTT ACC AAA
GTC CAC ACG GAA TGC TGC CAT GGA GAT CTG CTT GAA TGT GCT GAT GAC
AGG GCG GAC CTT GCC AAG TAT ATC TGT GAA AAT CAA GAT TCG ATC TCC
AGT AAA CTG AAG GAA TGC TGT GAA AAA CCT CTG TTG GAA AAA TCC CAC
TGC ATT GCC GAA GTG GAA AAT GAT GAG ATG CCT GCT GAC TTG CCT TCA
TTA GCT GCT GAT TTT GTT GAA AGT AAG GAT GTT TGC AAA AAC TAT GCT
GAG GCA AAG GAT GTC TTC CTG GGC ATG TTT TTG TAT GAA TAT GCA AGA
AGG CAT CCT GAT TAC TCT GTC GTG CTG CTG CTG AGA CTT GCC AAG ACA
TAT GAA ACC ACT CTA GAG AAG TGC TGT GCC GCT GCA GAT CCT CAT GAA
TGC TAT GCC AAA GTG TTC GAT GAA TTT AAA CCT CTT GTG GAA GAG CCT
CAG AAT TTA ATC AAA CAA AAT TGT GAG CTT TTT GAG CAG CTT GGA GAG
TAC AAA TTC CAG AAT GCG CTA TTA GTT CGT TAC ACC AAG AAA GTA CCC
CAA GTG TCA ACT CCA ACT CTT GTA GAG GTC TCA AGA AAC CTA GGA AAA
GTG GGC AGC AAA TGT TGT AAA CAT CCT GAA GCA AAA AGA ATG CCC TGT
GCA GAA GAC TAT CTA TCC GTG GTC CTG AAC CAG TTA TGT GTG TTG CAT
GAG AAA ACG CCA GTA AGT GAC AGA GTC ACC AAA TGC TGC ACA GAA TCC
TTG GTG AAC AGG CGA CCA TGC TTT TCA GCT CTG GAA GTC GAT GAA ACA
TAC GTT CCC AAA GAG TTT AAT GCT GAA ACA TTC ACC TTC CAT GCA GAT
ATA TGC ACA CTT TCT GAG AAG GAG AGA CAA ATC AAG AAA CAA ACT GCA
CTT GTT GAG CTC GTG AAA CAC AAG CCC AAG GCA ACA AAA GAG CAA CTG
AAA GCT GTT ATG GAT GAT TTC GCA GCT TTT GTA GAG AAG TGC TGC AAG
GCT GAC GAT AAG GAG ACC TGC TTT GCC GAG GAG GGT AAA AAA CTT GTT
GCT GCA AGT CAA GCT GCC TTA GGC TTA TAA
SEQ ID NO: 2 - Amino acid Sequence of the secreted Human Serum Albumin (585 amino
acid residues)
DAHKSEVAHRFKDLGEENFKALVLIAFAQYLQQCPFEDHVKLVNEVTEFAKTCVADESAENC
DKSLHTLFGDKLCTVATLRETYGEMADCCAKQEPERNECFLQHKDDNPNLPRLVRPEVDVMC
TAFHDNEETFLKKYLYEIARRHPYFYAPELLFFAKRYKAAFTECCQAADKAACLLPKLDELR
DEGKASSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
SEQ ID NO: 3 - Forward Primer
TCTGTCGACAAAAGAAGGGGTGTGTTTCGTCGAGATGCA
SEQ ID NO: 4 - Reverse Primer
ATGGAATTCATGTTATAAGCCTAAGGCAGCTTGACTTGC
SEQ ID NO: 5 - Nucleotide Sequence of Pro-Human Serum Albumin Gene (1776 base
pairs)
AGG GGT GTG TTT CGT CGA GAT GCA CAC AAG AGT GAG GTT GCT CAT CGG
TTT AAA GAT TTG GGA GAA GAA AAT TTC AAA GCC TTG GTG TTG ATT GCC
TTT GCT CAG TAT CTT CAG CAG TGT CCA TTT GAA GAT CAT GTA AAA TTA
GTG AAT GAA GTA ACT GAA TTT GCA AAA ACA TGT GTT GCT GAT GAG TCA
GCT GAA AAT TGT GAC AAA TCA CTT CAT ACC CTT TTT GGA GAC AAA TTA
TGC ACA GTT GCA ACT CTT CGT GAA ACC TAT GGT GAA ATG GCT GAC TGC
TGT GCA AAA CAA GAA CCT GAG AGA AAT GAA TGC TTC TTG CAA CAC AAA
GAT GAC AAC CCA AAC CTC CCC CGA TTG GTG AGA CCA GAG GTT GAT GTG
ATG TGC ACT GCT TTT CAT GAC AAT GAA GAG ACA TTT TTG AAA AAA TAC
TTA TAT GAA ATT GCC AGA AGA CAT CCT TAC TTT TAT GCC CCG GAA CTC
CTT TTC TTT GCT AAA AGG TAT AAA GCT GCT TTT ACA GAA TGT TGC CAA
GCT GCT GAT AAA GCT GCC TGC CTG TTG CCA AAG CTC GAT GAA CTT CGG
GAT GAA GGG AAG GCT TCG TCT GCC AAA CAG AGA CTC AAG TGT GCC AGT
CTC CAA AAA TTT GGA GAA AGA GCT TTC AAA GCA TGG GCA GTA GCT CGC
CTG AGC CAG AGA TTT CCC AAA GCT GAG TTT GCA GAA GTT TCC AAG TTA
GTG ACA GAT CTT ACC AAA GTC CAC ACG GAA TGC TGC CAT GGA GAT CTG
CTT GAA TGT GCT GAT GAC AGG GCG GAC CTT GCC AAG TAT ATC TGT GAA
AAT CAA GAT TCG ATC TCC AGT AAA CTG AAG GAA TGC TGT GAA AAA CCT
CTG TTG GAA AAA TCC CAC TGC ATT GCC GAA GTG GAA AAT GAT GAG ATG
CCT GCT GAC TTG CCT TCA TTA GCT GCT GAT TTT GTT GAA AGT AAG GAT
GTT TGC AAA AAC TAT GCT GAG GCA AAG GAT GTC TTC CTG GGC ATG TTT
TTG TAT GAA TAT GCA AGA AGG CAT CCT GAT TAC TCT GTC GTG CTG CTG
CTG AGA CTT GCC AAG ACA TAT GAA ACC ACT CTA GAG AAG TGC TGT GCC
GCT GCA GAT CCT CAT GAA TGC TAT GCC AAA GTG TTC GAT GAA TTT AAA
CCT CTT GTG GAA GAG CCT CAG AAT TTA ATC AAA CAA AAT TGT GAG CTT
TTT GAG CAG CTT GGA GAG TAC AAA TTC CAG AAT GCG CTA TTA GTT CGT
TAC ACC AAG AAA GTA CCC CAA GTG TCA ACT CCA ACT CTT GTA GAG GTC
TCA AGA AAC CTA GGA AAA GTG GGC AGC AAA TGT TGT AAA CAT CCT GAA
GCA AAA AGA ATG CCC TGT GCA GAA GAC TAT CTA TCC GTG GTC CTG AAC
CAG TTA TGT GTG TTG CAT GAG AAA ACG CCA GTA AGT GAC AGA GTC ACC
AAA TGC TGC ACA GAA TCC TTG GTG AAC AGG CGA CCA TGC TTT TCA GCT
CTG GAA GTC GAT GAA ACA TAC GTT CCC AAA GAG TTT AAT GCT GAA ACA
TTC ACC TTC CAT GCA GAT ATA TGC ACA CTT TCT GAG AAG GAG AGA CAA
ATC AAG AAA CAA ACT GCA CTT GTT GAG CTC GTG AAA CAC AAG CCC AAG
GCA ACA AAA GAG CAA CTG AAA GCT GTT ATG GAT GAT TTC GCA GCT TTT
GTA GAG AAG TGC TGC AAG GCT GAC GAT AAG GAG ACC TGC TTT GCC GAG
GAG GGT AAA AAA CTT GTT GCT GCA AGT CAA GCT GCC TTA GGC TTA TAA
SEQ ID NO: 6 - Amino acid Sequence of the Pro-Human Serum Albumin (591 amino acid
residues)
RGVFRRDAHKSEVAHRFKDLGEENFKALVLIAFAQYLQQCPFEDHVKLVNEVTEFAKTCVAD
ESAENCDKSLHTLFGDKLCTVATLRETYGEMADCCAKQEPERNECFLQHKDDNPNLPRLVRP
EVDVMCTAFHDNEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEVSKLVTDLTKVH
TECCHGDLLECADDRADLAKYICENQDSISSKLKECCEKPLLEKSHCIAEVENDEMPADLPS
LAADFVESKDVCKNYAEAKDVFLGMFLYEYARRHPDYSVVLLLRLAKTYETTLEKCCAAADP
HECYAKVFDEFKPLVEEPQNLIKQNCELFEQLGEYKFQNALLVRYTKKVPQVSTPTLVEVSR
NLGKVGSKCCKHPEAKRMPCAEDYLSVVLNQLCVLHEKTPVSDRVTKCCTESLVNRRPCFSA
LEVDETYVPKEFNAETFTFHADICTLSEKERQIKKQTALVELVKHKPKATKEQLKAVMDDFA
APVEKCCKADDKETCFAEEGKKLVAASQAALGL
SEQUENCE ID NO: 7 - Modified XhoI restriction site
CTCGAC

### DEFINITIONS

Unless defined otherwise, all technical and scientific terms used herein have the same meaning as commonly understood by one of ordinary skill in the art to which the methods belong. Although any vectors, host cells, methods and compositions similar or equivalent to those described herein can also be used in the practice or testing of the vectors, host cells, methods and compositions, representative illustrations are now described.

Where a range of values is provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The term “host cell” includes an individual cell or cell culture which can be, or has been, a recipient for the subject of expression constructs. Host cells include progeny of a single host cell. Host cell for the purposes of this disclosure refers to any strain of Pichia pastoris which can be suitably used for the purposes of the disclosure.

The term “recombinant strain” or “recombinant host cell” refers to a host cell which has been transfected or transformed with the expression constructs or vectors of this disclosure.

The term “expression vector” refers to any vector, plasmid or vehicle designed to enable the expression of an inserted nucleic acid sequence following transformation into the host.

The term “promoter” refers a DNA sequences that define where transcription of a gene begins. Promoter sequences are typically located directly upstream or at the 5′ end of the transcription initiation site. RNA polymerase and the necessary transcription factors bind to the promoter sequence and initiate transcription. Promoters can either be constitutive or inducible promoters. Constitutive promoters are the promoter which allows continual transcription of its associated genes as their expression is normally not conditioned by environmental and developmental factors. Constitutive promoters are very useful tool in genetic engineering because constitutive promoters drive gene expression under inducer-free conditions and often show better characteristics than commonly used inducible promoters. Inducible promoter are the promoters that are induced by the presence or absence of biotic or abiotic and chemical or physical factors. Inducible promoters are a very powerful tool in genetic engineering because the expression of genes operably linked to them can be turned on or off at certain stages of development or growth of an organism or in a particular tissue or cells.

The term “transcription” refers the process of making an RNA copy of a gene sequence. This copy, called a messenger RNA (mRNA) molecule, leaves the cell nucleus and enters the cytoplasm, where it directs the synthesis of the protein, which it encodes.

The term “translation” refers the process of translating the sequence of a messenger RNA (mRNA) molecule to a sequence of amino acids during protein synthesis. The genetic code describes the relationship between the sequence of base pairs in a gene and the corresponding amino acid sequence that it encodes. In the cell cytoplasm, the ribosome reads the sequence of the mRNA in groups of three bases to assemble the protein.

The term “expression” refers to the biological production of a product encoded by a coding sequence. In most cases a DNA sequence, including the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

The term “modified nucleic acid” as used herein is used to refer to a nucleic acid encoding human serum albumin. In a preferred embodiment, the modified nucleic acid is represented by SEQ ID NO:5 or a functionally equivalent variant thereof. Functional variant includes any nucleic acid having substantial or significant sequence identity or similarity to SEQ ID NO:5, and which retains the biological activity of the SEQ ID NO: 5.

The terms “polypeptide”, “peptide” and “protein” are used interchangeably herein to refer to two or more amino acid residues joined to each other by peptide bonds or modified peptide bonds. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. “Polypeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. “Amino acid” includes imino acid residues such as proline and hydroxyproline. The side chains may be in either the (R) or the (S) configuration.

### DETAILED DESCRIPTION OF THE INVENTION

The present disclosure discloses vectors and recombinant host cells for efficient production of biologically active and soluble recombinant human serum albumin (rHSA) as a secreted protein. Further, the disclosure provides a process for commercial scale production of recombinant human serum albumin.

The disclosure contemplates a multidimensional approach for achieving a high yield of recombinant human serum albumin in a heterologous host. The native gene for human serum albumin encodes for 609 amino acid long pre-pro albumin. The pre-pro albumin contains an 18 amino acid pre-domain (MKWVTFISLLFLFSSAYS), a 6 amino acid pro-domain (RGVFRR) followed by 585 amino acid human serum albumin protein. The pre-domain is processed by the endoplasmic reticulum and the pro-domain is processed in the Golgi apparatus. The inventors have developed a modified pPIC9 expression vector containing the 6 amino acid pro-domain (RGVFRR) followed by 585 amino acid HSA protein, without the pre-domain. Further, pPIC9 vector contains the restriction sites for both SalI and XhoI. To overcome the problems of self-ligation, the restriction site for XhoI was modified by ligating it with a SalI recognition sites to create a unique site (CTCGAC) containing the TCGA overhang. This unique site cannot be recognized by either XhoI or SalI enzymes.

The deletion of the pre-domain in combination with the use of modified vector, host cell and modified process parameters has resulted in an unprecedented enhancement in terms of yield.

In one embodiment, the modified nucleic acid is represented by SEQ ID NO: 5. The modified nucleic acid encodes pro human serum albumin (6 amino acid pro-domain followed by 585 amino acids). The pro-human serum albumin is represented by SEQ ID NO: 6.

The nucleic acid sequence of the secreted human serum albumin is SEQ ID NO: 1 and the secreted human serum albumin is represented by SEQ ID NO: 2.

In another embodiment, the modified nucleic acid is cloned in a yeast expression vector pPIC9.

In yet another embodiment, the pPIC9 expression vector contains a modified XhoI restriction site. The modified XhoI restriction site was modified by ligating it with a SalI recognition site to create a unique site (CTCGAC) containing the TCGA overhang. This unique site cannot be recognized by either XhoI or SalI enzymes.

In another embodiment, the process for cloning and expression of rHSA comprises the steps of cloning a modified nucleic acid (SEQ ID NO: 5) in yeast expression vector pPIC9 at XhoI/EcoRI site in-frame with the α-factor secretion signal sequence of Pichia pastoris present in pPIC9, followed by transforming the cloned vector into Pichia pastoris host cells, culturing said transformed Pichia pastoris cells to produce HSA and subsequently recovery and purification of recombinant HSA.

The expression of the HSA gene of interest is preferably driven by an AOX1 promoter, which is induced by methanol and repressed by glucose.

The strategy followed by preparation of the modified vector facilitates increases the efficiency in post-translational modification of the peptide which leads to enhanced secretion of the mature HSA protein into the extra-cellular medium.

In an embodiment, the expression vector containing the modified gene of interest (SEQ ID NO: 5) is transformed in an appropriate host.

In another embodiment, the expression vector containing the HSA gene of interest is transformed in yeast cells, preferably Pichia pastoris cells.

In a preferred embodiment, expression, the expression vector containing the HSA gene of interest is transformed in Pichia pastoris GS115 vectors.

In an embodiment, the process for production of recombinant human serum albumin is provided.

Aspects of present disclosure relates to fermentation of recombinant Pichia pastoris cells containing modified recombinant human serum albumin gene. After completion of the fermentation, the broth is centrifuged and the supernatant containing the human serum albumin is separated.

Accordingly, the process of production includes the steps of culturing recombinant host cells engineered for rHSA expression in claim in a suitable culture medium, harvesting the fermentation broth, followed by recovering and purifying recombinant human serum albumin from fermentation broth.

In one embodiment, the recombinant host cell is Pichia pastoris.

The medium (SBL medium) used for fermentation of Pichia pastoris containing rHSA gene of interest is selected from the group comprising Wegners Media (Wegner 1983). Basal salts BSM (In Vitrogen), FM 22 (Stratton 1998), YP medium (1% yeast extract, 2% Peptone) and YNB medium (YP with yeast Nitrogen base). Media composition used for cultivation of host cells is known to influence bio-process development by affecting production yields. Optimized cultivation parameters included addition of nitrogen source, pH, temperature, biomass at induction, duration of induction—phase. Specific components viz., (Biotin, YNB and ammonium sulphate) were optimized to obtain high yields in shorter periods. Process of preparation and the composition in the disclosure is explained in detail in Example 5.

The process of fermentation begins with inoculation of seed in the range of 3.5% to 5.5% v/v into SBL-PP medium selected from the media as described above. Prior to addition of seed, the fermenter is made ready by calibrating different probes like pH, DO etc. pH probe is calibrated using standard pH 4.0 and pH 7.0 solutions. Dissolved oxygen (DO) probe is one point calibrated with air for 100% DO. Initially DO is adjusted to 100%. DO is maintained above 20% by adjusting agitator speed and oxygen flow.

Generally, Pichia pastoris is optimally grown at about pH 4.8 to about pH 5.2. Between this pH range Pichia pastoris provided with a suitable nutrient media exhibits robust growth. This pH range also appeared to provide high levels of expression with human serum albumin (HSA). Further, in this process, fermentation of Pichia pastoris cells containing recombinant human serum albumin gene may be carried out in a 15 L fermenter. Scaling up of the process is done in a 55 L fermenter containing 24 to 26 L SBL-PP medium. The working volume of fermenter is always kept up to half the capacity of the fermenter.

In one embodiment, the percentage of inoculum or starter culture to initiate the fermenter culture is in the range of 3.5% to 5.5% v/v.

In another embodiment, the pH of the fermentation medium is maintained in the range of 5.0 to 5.8 as the secreted human serum albumin undergoes proper folding and is biologically active at this pH range.

In yet another embodiment, the temperature of the fermentation process is in the range of 28.5° C. to 30.5° C.

In another embodiment, the time for fermentation process is in the range of 90-140 hrs.

In a further, embodiment, the fermentation broth is centrifuged at a speed in the range from 10000 g to 11000 g, preferably 10540 g for a time period of about 8-12 min, preferably 10 min for separating the host cell and harvesting the supernatant.

The supernatant obtained after centrifugation is subjected to filtration and purified using ion-exchange chromatography to recover biologically active recombinant human serum albumin.

In one embodiment, the supernatant obtained after centrifugation is concentrated using a Tangential Flow Filtration System.

The size of the Tangential Flow Filtration (TFF) systems that may be used to concentrate the collected culture supernatant may range between 10 to 30 Kda (Sartorius TFF system).

The concentrated supernatant containing recombinant HSA was further subjected to ion exchange chromatography to recover biologically active recombinant HSA.

The chromatographic column for HSA purification is selected from DEAE Sepharose Ion Exchanger, CM Sepharose and Blue Sepharose.

Amino acid sequence analysis of the human serum albumin produced as per present disclosure was based on the Edman degradation method.

The first 15 N-terminal amino acid residues match with that of known plasma derived HSA. The remaining amino acids have been deduced from nucleotide sequence which shows 100% match with that of known plasma derived HSA.

The human serum albumin concentration obtained in this disclosure is found to be in the range of 1.4-1.7 gm/L. The recovery of rHSA after ion exchange chromatography (DEAE-column 1, 2) is in the range of 60-70% and the purity is about 98-99%.

### Examples

The following examples particularly describe the manner in which the disclosure is to be performed. But the embodiments disclosed herein do not limit the scope of the invention in any manner.

### Example 1: Isolation of Human Serum Albumin Gene

The human serum albumin gene was obtained by isolating RNA from human liver cells, synthesizing a first strand of cDNA there from by treating the same with a reverse primer of 5′-ATGGAATTCATGTTATAAGCCTAAGGCAGCTTGACTTGC-3′ as represented by SEQ ID NO:4 and then with a forward primer of 5′-TCTGTCGACAAAAGAAGGGGTGTGTTTCGTCGAGATGCA-3′ as represented by SEQ ID NO:3.

The nucleotide sequence of the cDNA of human serum albumin is represented by SEQ ID NO:1 and the amino acid sequence of human serum albumin is represented by SEQ ID NO:2.

For the purpose of the present disclosure, the human serum albumin cDNA was ligated at the SmaI site of pBSSK(+) plasmid and then transformed into TOP10™ E. coli cells for confirming the cDNA stretches.

### Example 2: Construction of Expression Vector Containing Human Serum Albumin Gene (pPIC9-HSA Clone)

The nucleic acid encoding human serum albumin was modified for recombinant expression by truncating the nucleic acid fragment encoding the 18 amino acid pre-domain (MKWVTFISLLFLFSSAYS) used in the prior art. The modified nucleic acid is represented by SEQ ID NO:5. The modified nucleic acid comprises 18 base pairs encoding the 6 amino acid pro-domain (RGVFRR), followed by 1758 base pairs encoding 585 amino acid mature human serum albumin protein.

The pro-human serum albumin protein encoded by the modified nucleic acid is represented by SEQ ID NO: 6.

A pPIC9 expression vector was designed by utilizing the modified nucleic acid represented by SEQ ID NO: 5. The forward primer (SEQ ID NO:3) containing the restriction site for XhoI enzyme and the reverse primer (SEQ. ID NO:4) containing the restriction site for EcoRI enzyme was used for ligating the modified nucleic acid (SEQ ID NO: 5). The modified nucleic acid was fused in frame with α-factor secretion signal of Pichia pastoris in the pPIC9 vector.

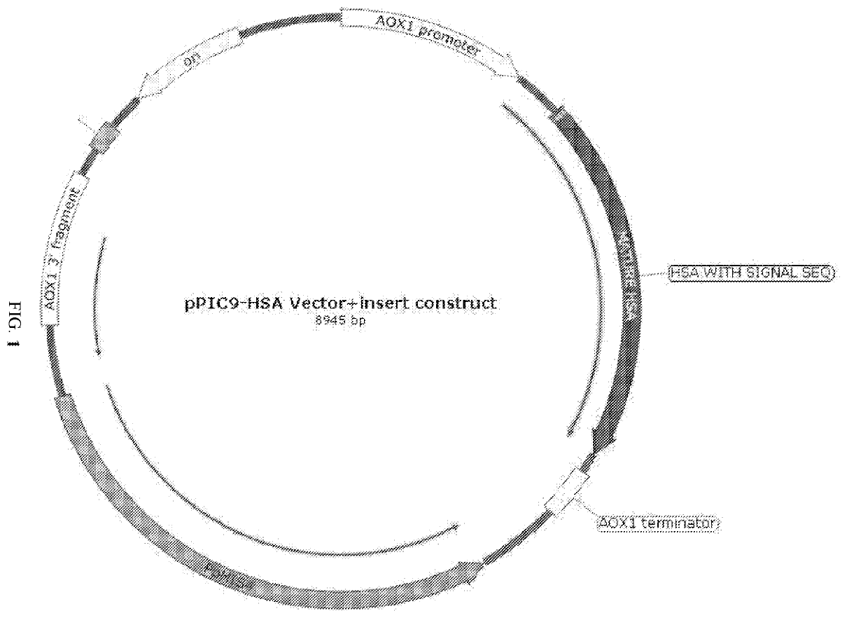

The construction scheme of pPIC9-HSA expression vector is depicted in FIG. 1. The pPIC9 expression vector used in the process contains the restriction sites for both XhoI (C/TCGAG) and SalI (G/TCGAC). The restriction site for XhoI was modified. XhoI site was modified by ligating it with a SalI recognition site to create a unique site (CTCGAC) containing the TCGA overhang. This unique site cannot be recognized by either XhoI or SalI enzymes.

The cloning and expression strategy adopted to get a new signal peptide facilitates cleavage of the signal peptide for human serum albumin with enhanced efficiency and facilitates enhanced secretion of the mature human serum albumin into the extra-cellular matrix.

### Example 3: PCR Amplification of the HSA Gene for Cloning and Expression in Pichia pastoris

PCR amplification of human serum albumin gene was performed with the conditions elaborated in Table 1.

TABLE 1
Conditions for PCR amplification of human serum albumin.

| Steps | Temperature | Time | | Cycles |
|---|---|---|---|---|
| Denaturation | 94° C. | 1 | minute | 30 cycles |
| Annealing | 55° C. | 1 | minute | |
| Extension | 72° C. | 1 | minute | |
| Final extension | 72° C. | 10 | minutes | — |

### Example 4: Expression of Recombinant Human Serum Albumin (HSA) Protein

Pichia pastoris GS115 strain was transformed with recombinant vector plasmid containing the HSA gene. The expression of the HSA gene was preferably driven by an AOX1 promoter, which is induced by methanol and repressed by glucose, thereby allowing high level of expression of the fused HSA gene.

The desired recombinant Human Serum Albumin protein is produced as a fusion product comprising 6 amino acid pro-domain (RGVFRR) followed by 585 amino acid mature human serum albumin protein. The 6 amino acid pro-domain are cleaved off in the Golgi apparatus during post-translational modification and the recombinant human serum albumin comprising the amino acid sequence of SEQ ID NO: 2 is released into the medium. Due to the absence of the 18 amino acid pre-domain, the post-translational processing is more efficient leading to a higher yield.

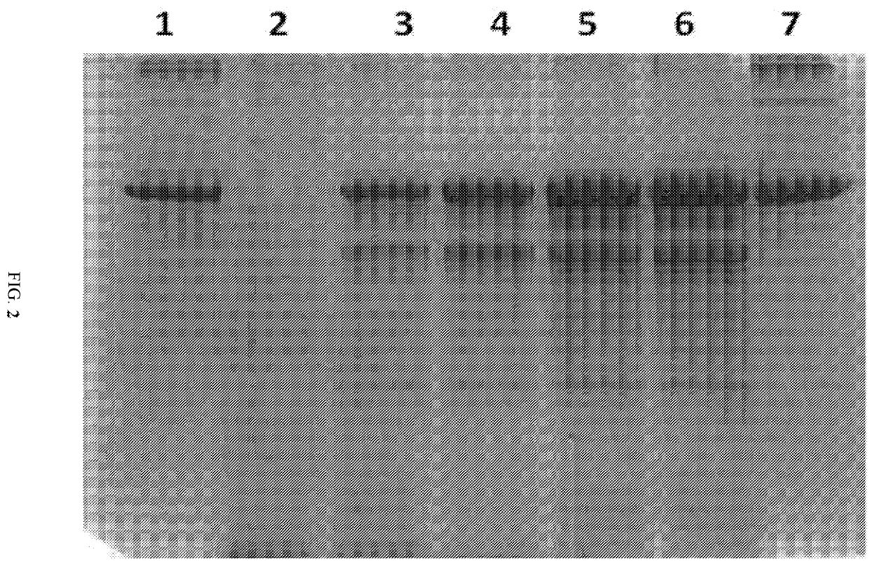

The expression of the recombinant human serum albumin was confirmed by SDS PAGE. The results of SDS PAGE are depicted in FIG. 2.

### Example 5: Fermentation of Recombinant Pichia pastoris Expressing Human Serum Albumin

Fermentation of recombinant Pichia pastoris cells containing modified human serum albumin gene (SEQ ID NO: 5) was carried out in 55 L fermenter with working volume of 24 L. Fermentation was carried out in SBL-PP medium as described herein using 4.58% inoculum as seed. The fermentation process lasted 96 hours and the whole process was carried out in three phases:
Phase 1: Preparation of SBL-PP medium
Phase 2: Preparation of seed inoculum
Phase 3: Process of fermentation

### Phase 1: Preparation of SBL Medium

The composition of SBL-PP medium optimized for the fermentation process is provided in Table 2.

TABLE 2
Composition of SBL-PP medium.

| Component | Concentration | |
|---|---|---|
| Yeast Extract | 10 | g/L |
| Peptone | 20 | g/L |
| Yeast Nitrogen Base | 3.4 | g/L |
| Glycerol | 41.6 | mL/L |
| Potassium dihydrogen phosphate | 10.2 | g/L |
| Dipotassium hydrogen phosphate | 4.29 | g/L |
| Biotin | 0.4 | mg/L |
| Ammonium Sulphate | 10 | g/L |

| | |
|---|---|
| Antifoam | 0.01% |

These components were calculated, weighed and dissolved for 24 L medium stepwise as follows:
(a) Yeast extract, peptone and antifoam were weighed and dissolved in required water and pumped into fermenter. The components were autoclaved at 15 lbs/sq. inch for 20 minutes at 121° C. and allowed media to cool to 30° C.
(b) Autoclaved 50% Glycerol solution and 1M Phosphate buffer solution were separately added to the fermenter.
(c) Autoclaved Ammonium sulphate, Filter Sterile YNB & biotin solution were added to the fermenter and the medium pH was adjusted to be in the range of 5.9 to 6.1.
(d) 1685 ml of Glycerol plus 1685 ml of water for injection (WFI) autoclaved at 121° C. for 20 minutes.
(e) PTM1 Trace salts were dissolved and made up to 1 L and filter sterilized through 0.2 micron filter. The PTM trace salts are elaborated in Table 3.

TABLE 3
PTM trace salts.

| | | |
|---|---|---|
| Cupric sulphate.5H₂O | 6 | g/l |
| Sodium Iodide | 0.08 | g/l |
| Manganese sulphate.H₂O | 3 | g/l |
| Sodium Molybdate2H₂O | 0.2 | g/l |
| Boric Acid | 0.02 | g/l |
| Cobalt chloride | 0.5 | g/l |
| Zinc Chloride | 20 | g/l |
| Ferrous sulphate.7H₂O | 65 | g/l |
| Biotin | 0.2 | g/1 |
| Sulphuric acid (concentrated) | 5 | ml/l |

(f) Cupric sulphate.5H₂O needs to be dissolved separately in 50 ml of WFI. Ferrous sulphate.7H₂O needs to be separately dissolved in WFI and heated up to 60 to 70° C. in water bath. Biotin is dissolved in 100 ml of WFI and kept for overnight stirring at 2 to 8° C.)
(g) 988 ml Methanol and 12 ml PTM1 salts solution were mixed to comprise 1 L of induction medium.
(h) Ammonia solution was used to adjust the pH during various phases of fermenter culture. Growth phase pH was maintained between 5.7-5.8. Induction phase pH was maintained between 5.7-5.8 up to 36 induced hours. Stationary phase pH was maintained between 5.0-5.2 beyond 36 induced hours till harvest stage.
(i) 9 ml antifoam sigma A5758 at a concentration of 3% was suspended in 300 ml of WFI and autoclaved at 121° C. for 20 minutes.

### Phase 2: Preparation of Seed Inoculum

For seeding 4.58% of SBL-PP medium (1100 ml of 24 L medium) was inoculated with 1.2 ml of glycerol stock from WCB in three 2 L conical flasks containing 400 ml medium. The flasks were kept at orbital shaker at 29° C., 200 rpm for 24-30 hours. Optical Density (OD₆₀₀) of the culture was read in spectrophotometer (manufactured by Shimadzu Co.).

### Phase 3: Fermentation Process

The whole process of fermentation begins with inoculation of seed at 4.58% in to SBL-PP medium. The pre-grown seed was inoculated into 17 L of autoclaved SBL-PP medium along with the seed (1.1 L). 2.0 L of 50% glycerol, 2.4 L of sterile phosphate buffer solution, 1 L of yeast nitrogen base (YNB), 600 ml of Ammonium sulphate and 48 ml of Biotin solution were also added through the inlet pump. Prior to addition of seed, the fermenter was made ready by calibrating different probes like pH, DO etc. as follows:

a) pH probe calibration: pH probe was calibrated using standard pH 4.0 and pH 7.0 solutions.
b) Dissolved oxygen (DO) probe calibration: Probe was one point calibrated with air for 100% DO.
Fermentation conditions: The fermentation parameters set were as given in Table 4 and the fermentation was started by quick addition of seed into inoculation port.

TABLE 4
Fermentation Parameters.

| | |
|---|---|
| Temperature | 29° C. |
| pH in growth phase | 5.7 to 5.8 |
| pH in induction phase 0-36 hours | 5.7 to 5.8 |
| pH in stationary phase 37 hours to end | 5.8 to 6.5 |
| Dissolved Oxygen | 20-90% |
| Air flow (LPM) | 1 to 6 |
| VVM | 0.25 |
| Back pressure | 0 |
| Agitation | 300 to 650 rpm |

Initially DO was adjusted to 100% and was maintained above 20% as described in the process.

### pH Monitoring During the Fermentation Process:

pH was monitored carefully during the process of fermentation from the seed inoculation stage till the end of fermentation. Initially, during the growth phase of Pichia cells, the pH of the broth drops to 5.5 and then pH was adjusted to 5.8 with ammonia solution. pH was maintained at 5.7 to 5.8 during growth phase. Samples of 1 mL were collected from the fermenter at different time points, viz., before glycerol feed, before induction, after induction every 12 hours interval till the end. Selected samples were processed for loading on to the gel for SDS-PAGE and endotoxin analysis. Also, the cells were observed under the microscope for the presence of any foreign organisms. After 24 hours of fermentation there was a spike in DO to 100% and optical density was around 62 and the biomass was 100 mg/mL. At this point, glycerol feed was initiated and pumped within 9 hours. At this stage cells were starved for 1-2 hours. After starvation period pH was in raising trend and OD was 160 and the biomass 230 mg/ml.

After starvation, methanol feeding was initiated. The rate of methanol feeding is given in Table 5.

TABLE 5
Rate of methanol feeding.

| Time | Rate | |
|---|---|---|
| 0 to 6 hours | 60 | ml/hour |
| 7 to 18 hours | 90 | ml/hour |
| 19 to 78 hours | 120 | ml/hour |
| 79 to 92 hours | 90 | ml/hour |
| 93 & 94 hours | 60 | ml/hour |
| 95 & 96 hours | 0 | ml/hour |

### Example 6: Cell Harvesting and Purification

The next step involves harvesting the cells. After running the fermenter for 96 to 100 hours following seed inoculation, the OD₆₀₀ of the fermenter sample was 230 OD/ml and biomass was found to be 330 mg/ml. The fermenter batch was terminated by switching off all the controls.

### Clarification of Fermentation Broth

After termination of the batch the broth was centrifuged at 10540 g for 10 minutes at 4° C. The supernatant was collected, and the conductivity was found to be 18 ms/cm and the pH was 5.5. The pellets were discarded.

### Concentration of Supernatant

The collected supernatant was concentrated to 14-fold with 30 KDa Tangential Flow Filtration system.

The TFF system parameters are as given below: (range)
(a) Temperature—25° C.
(b) Inlet pressure—2 bar
(c) Outlet pressure—1.8 bar
(d) Flow rate—120 ml/min

### Buffer Exchange Through TFF

2 L concentrated supernatant was made up to 28 L (Initial supernatant volume) with 20 mM phosphate buffer pH 6.5 and the said sample was equilibrated for 30 min at RT. The concentration step was repeated. After buffer exchange, 2.6 L was obtained, and the pH was 6.6 and the conductivity was 2.9 ms/cm.

### Centrifugation of the Buffer Exchanged Sample

After buffer exchange, the sample was centrifuged at 10540 g for 30 minutes at 4° C. The supernatant was collected, and the pellet was discarded.

### Filtration of the Sample

The supernatant obtained after centrifugation was passed through 0.4 microns nylon membrane filter and membrane was rinsed with 20 mM phosphate buffer pH 6.5 for complete recovery.

### Example 7: Purification Using Ion Exchange Chromatography and Yield

Four major steps are followed for protein purification:

### 1) Equilibration

### 2) Sample Preparation

### 3) Binding or Capture

### 4) Elution

A two-stage purification process using ion exchange chromatography was performed. The Ion Exchange Chromatography parameters are given below:
(a) System-AKTA Purifier UPC 100 (GE Health care)
(b) Column-Index column 140/500
(c) Matrix-DEAE Sepharose
(d) Bed volume-3.0 L

The AKTA system was switched on and step by step operations were followed as given below:

1) Column washed with 0.2 microns filtered water (3 column volumes) at 50 mL/min flow rate
2) Column equilibrated with 20 mM sodium phosphate buffer pH 6.5 (10 column volumes)
3) 3.0 L Sample loaded on to the column at a flow rate of 17 mL/min
4) The UV, Conductivity and pH values were monitored
5) 3.0 L flow through was collected
6) After sample application, the unbound material was eluted with 1 column volume (CV) of equilibration buffer
7) Sample was eluted with a linear gradient of increasing NaCl concentration (0-100%) in elution buffer (10CV) and the elution buffer as given below:
Buffer A contained 20 mM sodium phosphate buffer pH 6.5 and 50 mM NaCl. Buffer B contained 20 mM sodium phosphate buffer pH 6.5 and 300 mM NaCl.

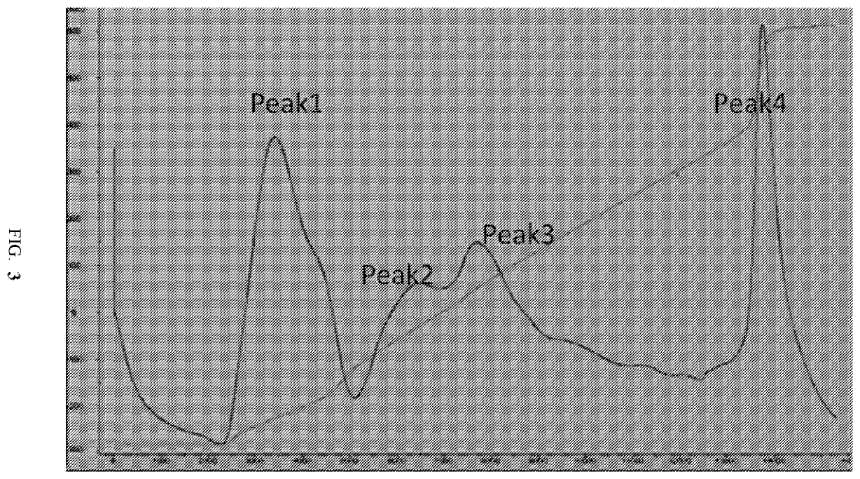

FIG. 3 depicts the elution profile of recombinant Human Serum Albumin from DEAE sepharose column. The sample was analyzed by SDS-PAGE for purity.

After elution the column cleaning in place (CIP) was done.

Thereafter, a second stage purification process was performed. The target peak obtained from column I (1700 ml) was diluted to 8 L until the conductivity was 3 ms/cm and pH 6.5.

The same ion exchange chromatography parameters as used in Example 1 were used.

The sample was again analyzed by SDS-PAGE for purity.

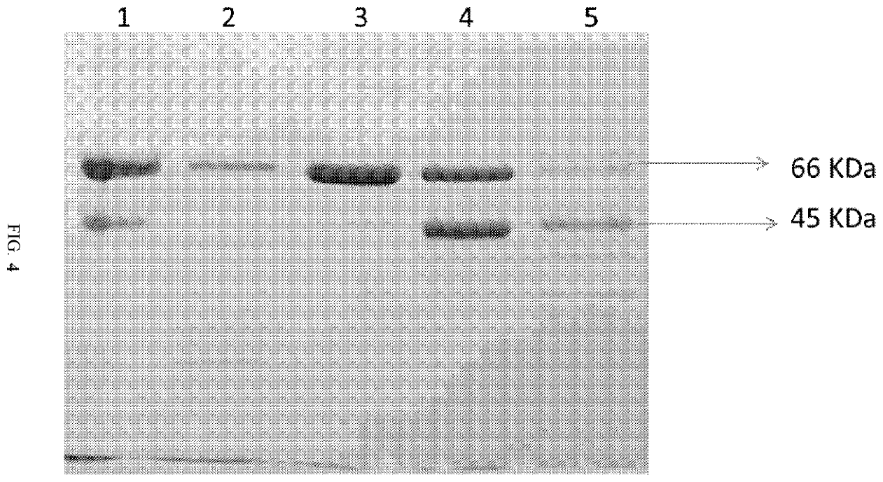

FIG. 4 depicts photomicrograph of native SDS-PAGE of rHSA after DEAE Sepharose column purification. Lane 1 depicts eluted sample peak fraction from DEAE sepharose column before loading. Lane 2 depicts Flow through sample. Lane 3 depicts peak 1 pooled fraction eluted from DEAE sepharose column. Lane 4 depicts peak 2 pooled fractions eluted from DEAE sepharose column. Lane 5 depicts peak 3 pooled fractions eluted from DEAE sepharose column.

The human serum albumin concentration was found to be 1.4-1.7 gm/L. In most of the batches, the concentration was 1.4-1.7 gm/L. The yield percentage of the recombinant human serum albumin was in the range of 60-70%. The purity of the recombinant human serum albumin was about 98-99%.

1. A pPIC9 expression vector comprising the nucleic acid of SEQ ID NO: 5, wherein the nucleic acid encodes human serum albumin.

2. The expression vector as claimed in claim 1, wherein the vector comprises a modified XhoI restriction site comprising the nucleotide sequence of SEQ ID NO: 7.

3. A recombinant host cell comprising the modified expression vector as claimed in claim 1.

4. The recombinant host cell as claimed in claim 3, wherein the recombinant host cell is Pichia pastoris.

5. A process for producing recombinant human serum albumin, comprising the steps of:
a. culturing recombinant host cells as claimed in claim 3 in a suitable fermentation medium to obtain a fermentation broth;
b. harvesting supernatant from the fermentation broth, wherein the supernatant contains recombinant human serum albumin; and
c. purifying recombinant human serum albumin.

6. The process as claimed in claim 5, wherein the recombinant host cell is Pichia pastoris.

7. The process as claimed in claim 5, wherein the fermentation medium is SBL-PP medium.

8. The process as claimed in claim 5, wherein the percentage of host cell inoculum in the fermentation medium is in the range from 3.5% to 5.5% v/v.

9. The process as claimed in claim 5, wherein the pH of the fermentation broth is maintained in the range from 5.0 to 5.8.

10. The process as claimed in claim 5, wherein the temperature of the fermentation broth in maintained in the range from 28.5° C. to 30.5° C.

11. The process as claimed in claim 5, wherein the time for culturing is in the range from 90 to 140 hr.

12. The process as claimed in claim 5, wherein the supernatant is harvested by centrifuging the fermentation broth at a speed in the range from 10000 to 11000 g for a period in the range from 8 to 12 min to recover the human serum albumin.

13. The process as claimed in claim 5, wherein the harvested recombinant human serum albumin is purified by tangential flow filtration and ion exchange chromatography.

14. The process as claimed in claim 13, wherein the chromatographic column used for ion exchange chromatography is selected from a group comprising DEAE Sepharose, CM Sepharose and Blue Sepharose.

15. A genetically engineered Pichia strain comprising a modified pPIC9 expression vector that does not encode the 18 amino acid pre-domain of human serum albumin gene.

16. The genetically engineered Pichia strain of claim 15, wherein the modified pPIC9 expression vector comprises a nucleic acid of SEQ ID NO: 5, wherein the nucleic acid encodes human serum albumin.

17. The genetically engineered Pichia strain of claim 16, wherein the modified pPIC9 expression vector comprises a modified XhoI restriction site comprising a nucleotide sequence of SEQ ID NO: 7.

18. The genetically engineered Pichia strain of claim 17, wherein the Pichia strain comprises recombinant Pichia pastoris host cells.

19. The genetically engineered Pichia strain of claim 18, wherein the recombinant Pichia pastoris host cells express recombinant human serum albumin as a secreted protein.

20. The genetically engineered Pichia strain of claim 19, wherein the secreted protein has a concentration in the range of 1.4 to 1.7 gm/L and a purity greater than 98%.
